(19) United States
(12) Patent Application Publication
(10) Pub. No.: US 2012/0041469 A1
(43) Pub. Date: Feb. 16, 2012

(54) REVASCULARIZATION DEVICE WITH INTEGRATED DISTAL EMBOLI PROTECTION

(75) Inventors: DAVID R. FISCHELL, FAIR HAVEN, NJ (US); TIM A. FISCHELL, KALAMAZOO, MI (US)

(73) Assignee: SVELTE MEDICAL SYSTEMS, INC., NEW PROVIDENCE, NJ (US)

(21) Appl. No.: 12/854,464

(22) Filed: Aug. 11, 2010

(51) Int. Cl. A61F 2/01

(52) U.S. Cl. 606/200

(57) ABSTRACT

A percutaneous system to open a stenosed vessel has a catheter for insertion into a vessel. An expandable filter mechanism is within a deployable sheath for expansion against a vessel wall when the sheath is displaced by a first displacement distance. A stenosis opening mechanism is within the deployable sheath and is radially expandable near the expandable filter mechanism with the stenosis opening mechanism being expandable against the stenosis when the stenosis opening mechanism is located within the vessel in longitudinal alignment with the stenosis following displacement of the sheath by a second displacement distance. Radiopaque markers align the stenosis opening mechanism with the stenosis.

### FIELD OF USE

This invention is in the field of percutaneous devices that are used to open a vessel of the human body.

### BACKGROUND OF THE INVENTION

In balloon angioplasty or stenting of vessels of the human body, embolic debris can be released and embolize down stream (distally). This can cause damage to tissue distal to the treatment site, including myocardial infarction when coronary arteries are treated, or strokes if carotid arteries are being treated, etc.. Existing distal emboli protection devices such as the Angiogard device of Cordis or RX Accunet of Abbott Laboratories are integrated into a guide wire. This has the disadvantage of having to be delivered to the site of the obstruction before the device being used for revascularization of the vessel is delivered. This mandates one or more extra steps in the procedure to deliver the revascularization device, and requires the use of one or more revascularization devices (extra device). On the other hand, it would be of significant advantage to be able to deliver a revascularization device that has a distal emboli protection capability integrated into the revascularization device itself.

### SUMMARY OF THE INVENTION

The present invention is the incorporation of a distal emboli collection device into the distal section of a percutaneously inserted device for opening an obstruction in a vessel of the human body. Such opening or “revascularization” devices include balloon angioplasty catheters, atherectomy catheters, lasers, and stents. Stents used for recannalization include balloon expandable stents such as the Cordis Cypher and Abbott Xience drug eluting stents and non drug eluting stents such as the Abbott Vision or Medtronic Driver stents and self-expanding stents like the Cordis PRECISE and SMART stents and the Abbott Acculink stent. As self-expanding stents almost always require post implant balloon dilatation, it is envisioned that one embodiment of the present invention would include a self-expanding stent delivery system with an integrated distal emboli protection capability, and an integrated angioplasty balloon for post dilatation, with all three capabilities contained in one device. Although the present invention can be configured as an over the wire or rapid exchange catheter using a standard guidewire, it is also envisioned that to reduce the profile, a fixed wire distal end such as that described by Fischell et al in U.S. Pat. Nos. 6,375,660, 6,936,065 and 7,011,673.

Thus it is an object of the present invention to provide the capability for both distal emboli protection and vessel opening in one device.

Another object of the present invention to have a balloon angioplasty catheter with an integrated distal emboli protection capability

Still another object of the present invention is to have a balloon expandable stent delivery system with integrated distal emboli protection capability.

Still another object of the present invention is to have a self-expanding stent delivery system with integrated distal emboli protection capability

Yet another object of the present invention is to have a self-expanding stent delivery system with integrated distal emboli protection and a built-in angioplasty balloon.

These and other objects and advantages of this invention will become obvious to a person of ordinary skill in this art upon reading of the detailed description of this invention including the associated drawings.

### BRIEF DESCRIPTION OF THE DRAWINGS

FIG. 1 is a longitudinal cross section of the distal end of the present invention fixed wire self expanding stent delivery system that includes a balloon to be used for post dilatation of a self expanding stent.

FIG. 2A is an enlargement of the longitudinal cross section of section 2A of the present invention fixed wire self expanding stent delivery system of FIG. 1

FIG. 2B is an enlargement of the longitudinal cross section of section 2B of the present invention fixed wire self expanding stent delivery system of FIG. 1

FIG. 2C is a longitudinal cross section of the proximal end of the stent delivery system of FIG. 1.

FIG. 3A is a longitudinal cross section of the distal section of a stent delivery system with integrated distal protection that is delivered over a guide wire.

FIG. 3B is a longitudinal cross section of the proximal end of an over the wire version of the balloon expandable stent delivery system of FIG. 3A

FIG. 3C is a longitudinal cross section of the central section of a rapid exchange version of the stent delivery system of FIG. 3

FIG. 4A is a longitudinal cross section of the fixed wire delivery system of FIG. 1 after it has been positioned at the site of a stenosis in a vessel of a human body.

FIG. 4B is a longitudinal cross section of the system of FIG. 1 after the sheath has been pulled back enough to deploy the distal protection subsystem.

FIG. 4C is a longitudinal cross section of the system of FIG. 1 after the sheath has been pulled back completely and the self expanding stent has been deployed

FIG. 4D is a longitudinal cross section of the system of FIG. 1 as it would appear during balloon inflation to post dilate the self expanding stent.

FIG. 4E is a longitudinal cross section of the system of FIG. 1 after the sheath has been advanced until it is ready to collapsed the distal protection sub system.

FIG. 4F is a longitudinal cross section of the system of FIG. 1 after the distal protection subsystem has been completely collapsed but before the system is withdrawn from the human body.

FIG. 4G is a longitudinal cross section of the vessel of the human body with the post dilated self expanding stent following removal of the stent delivery system.

FIG. 5 is a longitudinal cross section of the distal end of the present invention fixed wire balloon angioplasty catheter with integrated distal emboli protection

FIG. 6 is a longitudinal cross section of the distal end of the present invention balloon angioplasty catheter with integrated distal emboli protection that is advanced over a guide wire.

FIG. 7 is a longitudinal cross section of the distal end of the present invention fixed wire self expanding stent delivery system with integrated distal emboli protection.

FIG. 8 is a longitudinal cross section of the distal end of the present invention self expanding stent delivery system with integrated distal emboli protection that is advanced over a guide wire.

### DETAILED DESCRIPTION OF THE DRAWINGS

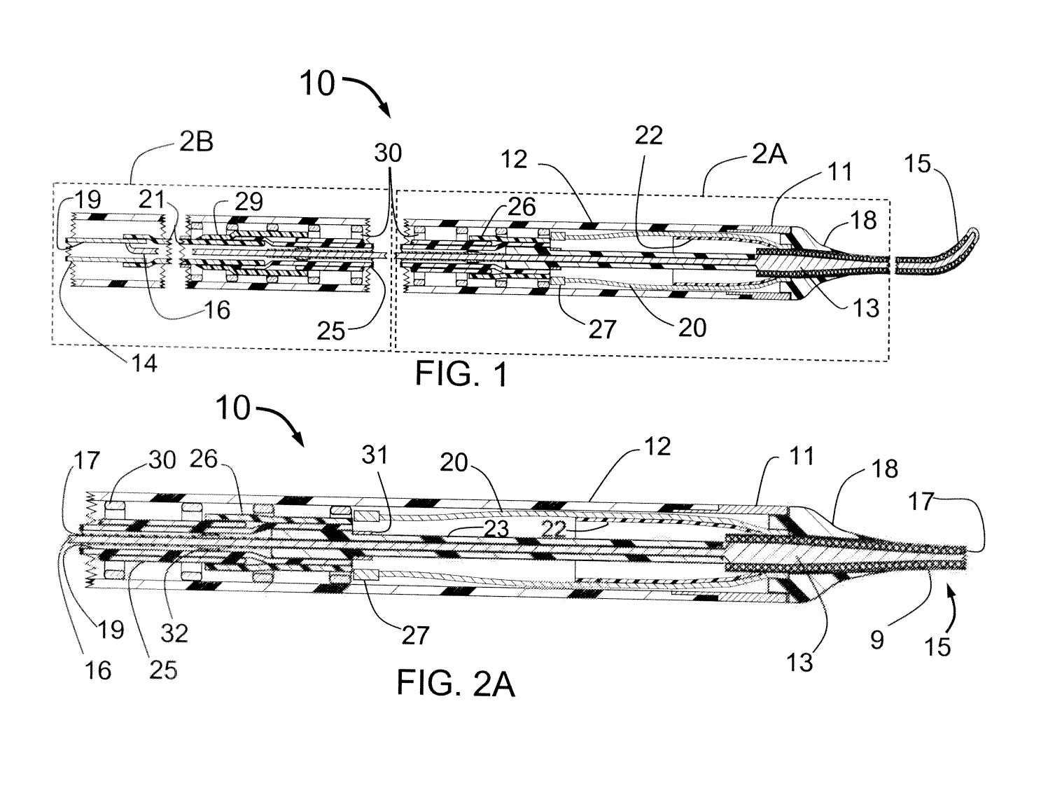

FIG. 1 is a longitudinal cross section of the distal end of the present invention system 10 for the opening of blocked vessels of the human body that includes both an expandable stent 30 and a balloon 25 to be used for expansion of the stent 30. The stent 30 shown here is a self expanding stent although the stent delivery system 10 could be used with a balloon expandable stent. If the stent 30 is a self-expanding stent then the balloon 25 is used only for post-dilatation. If the stent 30 is a balloon expandable stent, then the balloon 25 can be used for both initial deployment and post-dilatation. The system 10 includes an integral fixed guide wire 15 with core wire 16 having a distal tapered section 13. The system 10 includes a deployment sheath 12 with metal radiopaque distal end 11 that fits over the distal nose piece 18 which is coaxially attached to the fixed guide wire 15. Under the sheath 12 at the just proximal to the distal nose piece 18 is an expandable embolic filter 22 attached to an expandable/retractable wire basket 20 whose distal end is in turn attached to a radiopaque ring 27. The embolic filter 22 is a porous material that allows blood to go through it but not embolic debris released during opening of the stenosis. Such embolic filters are currently in use mounted on guide wires and are typically constructed from fabric or plastic. Under the sheath 12, a stent 30 is coaxially located over an angioplasty balloon 25. The self expanding stent 30 in this embodiment is longer than the cylindrical portion of the balloon 25 which is positioned to dilate the central portion of the stent after deployment. The stent 30 and balloon 25 are located proximal to the radiopaque ring 27 at the proximal end of the wire basket 20. Either or both the stent 30 and wire basket 20 may have an anti-thrombogenic coating such as carbon. Elastic distal balloon control band 26 and proximal balloon control band 29 placed coaxially over the ends of the balloon 25 act to refold the angioplasty balloon 25 after inflation. The proximal end of the balloon 25 is coaxially mounted onto the distal end of the plastic tube 21. The proximal end of the plastic tube 21 is coaxially mounted onto a metal hypotube 14 with balloon inflation lumen 19. The metal hypotube 14 is typically a thin wall stainless steel tube. The proximal end of the core wire 16 is attached (typically welded) to the hypotube 14

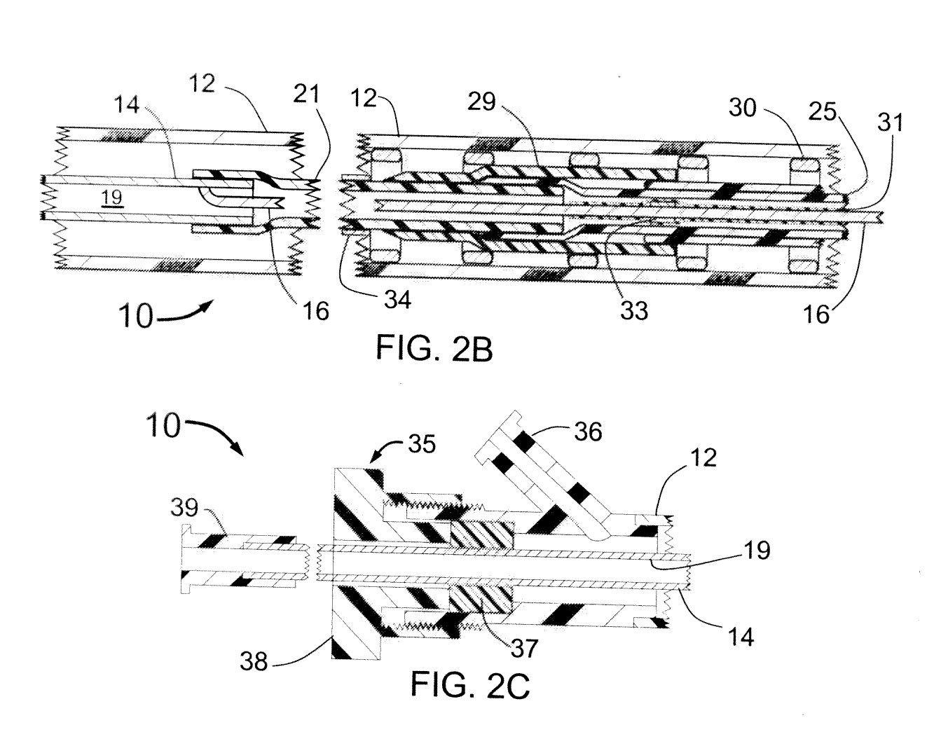

FIG. 2A is an enlargement of the longitudinal cross section of section 2A of the present invention fixed wire self expanding stent delivery system 10 of FIG. 1. The integral fixed guide wire 15 with core wire 16 having a distal tapered section 13 includes a coaxially wrapped helical wire coil 17 with plastic coating 9. The entire catheter 10 or just the tip would be typically lubricity coated. The system 10 includes a deployment sheath 12 with metal radiopaque distal end 11 that fits over the distal nose piece 18 which is coaxially attached to the plastic coating 9 of the fixed guide wire 15. Under the sheath 12 at the just proximal to the distal nose piece 18 is an expandable embolic filter 22 attached to an expandable/retractable wire basket 20 whose distal end is in turn attached to a radiopaque ring 27. A plastic member 23 is attached to the core wire 16 under and extending proximally from the wire basket 20. The angioplasty balloon 25 with balloon control band 26 is coaxially mounted onto the proximal end of the member 23. Under the sheath 12, a stent 30 is coaxially located over an angioplasty balloon 25. The stent 30 and balloon 25 are located proximal to the radiopaque ring 27 at the proximal end of the wire basket 20. The stent distal radiopaque marker band 31 is mounted coaxially over the member 23 at a location just distal to the distal end of the stent 30. The balloon distal radiopaque marker band 32 is mounted coaxially onto the core wire 16 and marks the distal end of the cylindrical portion of the balloon 25 when expanded. A plastic tube 33 is shrunk over the marker band 32 and core wire 16 so as to protect the inside of the folded balloon 25 from coming in contact with the metallic band 32. The space between the angioplasty balloon 25 and the shrunk plastic tube 33 is the balloon inflation lumen 19.

FIG. 2B is an enlargement of the longitudinal cross section of section 2B of the present invention fixed wire self expanding stent delivery system 10 with sheath 12 of FIG. 1. FIG. 2B shows the proximal ends of the stent 30 and angioplasty balloon 25 with proximal control band 29. The proximal end of the balloon 25 is coaxially mounted onto the distal end of the plastic tube 21. The balloon proximal radiopaque marker band 33 is mounted coaxially onto the core wire 16 and marks the proximal end of the cylindrical portion of the balloon 25 when expanded. An optional plastic tube 17 is shrunk over the marker band 33 and core wire 16 so as to protect the inside of the folded balloon 25 from coming in contact with the metallic band 33. The stent proximal radiopaque marker band 34 is mounted coaxially over the plastic tube 21 at a location just proximal to the proximal end of the stent 30. The proximal end of the plastic tube 21 is coaxially mounted onto the hypotube 14 with balloon inflation lumen 19. The proximal end of the core wire 16 is attached (typically welded) to the hypotube 14 near its distal end.

FIG. 2C is a longitudinal cross section of the proximal end of the stent delivery system 10 of FIG. 1. The sheath 12 is coaxially mounted onto the distal end of the Tuohy-Borst fitting 35 with side port 36, proximal hub 38 and elastomer sealing ring 37. The proximal luer fitting 39 for balloon inflation is attached to the hypotube 14 and in fluid communication with the balloon inflation lumen 19. With the Tuohy-Borst fitting 35 tightened over the hypotube 14, no fluid such as blood will leak out of the system 10. When the Tuohy-Borst fitting is loosened and slid in the proximal direction it will cause the sheath 12 to slide back releasing the wire basket 20 and filter 22 of FIG. 2A. Subsequent proximal movement of the Tuohy-Borst 35 and sheath 12 will slide back over the ring 27 and collapse the wire basket 20 so the system 10 can be removed from the body.

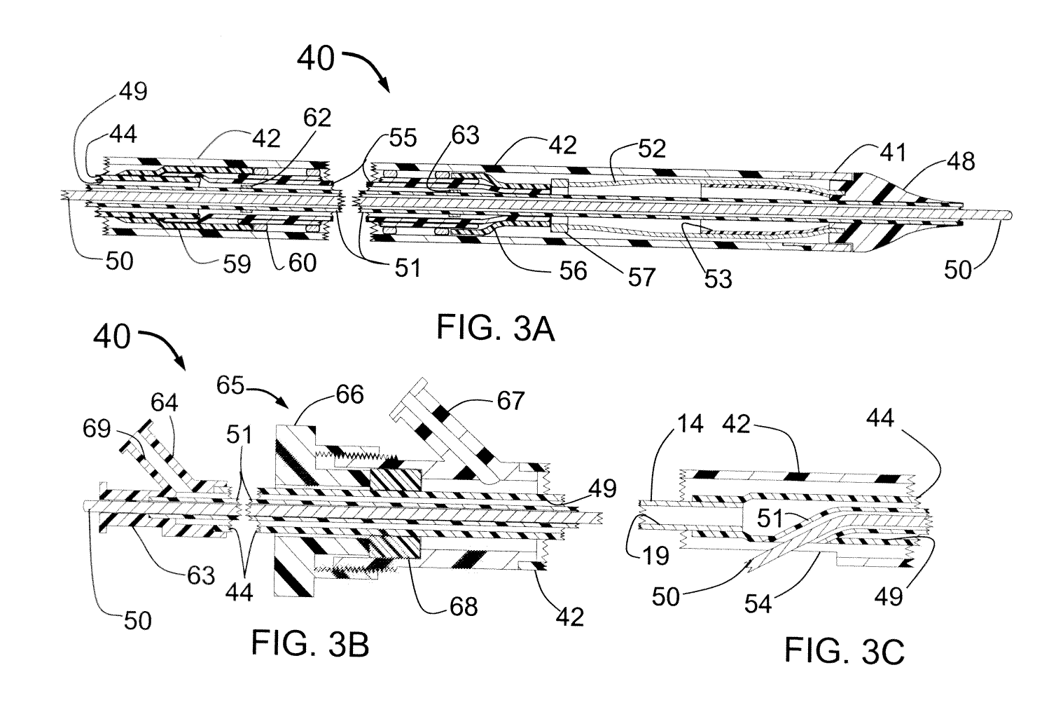

FIG. 3A is a longitudinal cross section of the distal section of a stent delivery system 40 with integrated distal protection that is delivered over a guide wire 50. The system 40 with stent 60 includes a sheath 42 with radiopaque metallic distal end 41 that fits over the distal nose piece 48 that is coaxially mounted over the guide wire tube 51. The system 40 includes an angioplasty balloon 55 to be used for expansion of the stent 30. The stent 60 shown here is a balloon expandable stent although the stent delivery system 40 could be used with a self expanding stent. If the stent 60 is a balloon expandable stent, then the balloon 55 can be used for both initial deployment and post-dilatation. If the stent 60 is a self-expanding stent then the balloon 55 is used only for post-dilatation. The balloon expandable stent 60 shown here is just a slight bit (e.g. 1-2 mm) shorter than the cylindrical portion of the balloon 55. With a self expanding stent, it may be desirable to have the stent 60 be longer than the cylindrical portion of the balloon 55 as is shown in FIG. 1. Under the sheath 42 at the just proximal to the distal nose piece 48 is an expandable embolic filter 53 attached to an expandable/retractable wire basket 52 whose distal end is in turn attached to a radiopaque ring 57. Under the sheath 42, the stent 60 is coaxially located over an angioplasty balloon 50. The balloon 55 is located proximal to the radiopaque ring 57 at the proximal end of the wire basket 52. The distal end of the balloon 55 is coaxially mounted onto the guide wire tube 51 at a location just proximal to the ring 57. The proximal end of the balloon 55 is coaxially mounted onto the distal end of the plastic tube 44. Elastic distal balloon control band 56 and proximal balloon control band 59 placed coaxially over the ends of the balloon 55 act to refold the angioplasty balloon 55 after inflation. The stent 60 is mounted between the control bands 56 and 59. A second advantage of the control bands 56 and 59 is that they help the balloon 55 inflate more uniformly to expand the stent 55 without first popping open at the ends which can cause injury to the vessel. The proximal end of the balloon 55 is coaxially mounted onto the distal end of the plastic tube 44. The space between the plastic tube 44 and the guide wire tube 51 is the balloon inflation lumen 49. Proximal and distal radiopaque marker bands 62 and 63 respectively mark the ends of the cylindrical portion of the balloon 55 and the balloon expandable stent 60. The system 40 can be either an over the wire or rapid exchange type system.

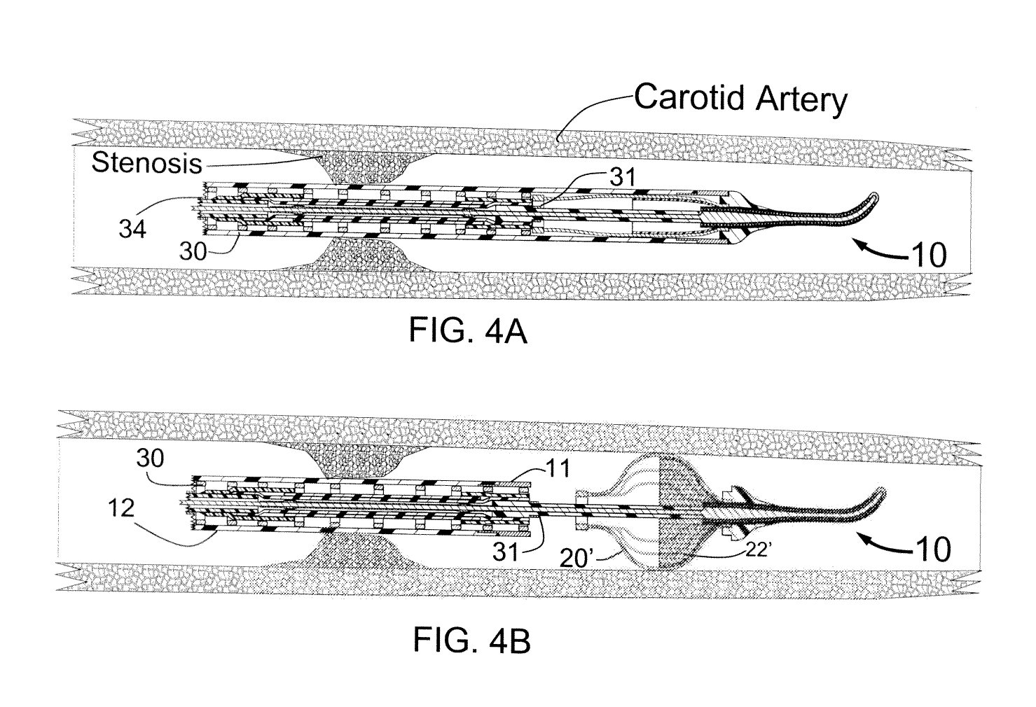

FIG. 3B shows a longitudinal cross section of the proximal end of the over the wire version of the system 40 of FIG. 4A. The sheath 42 is coaxially mounted onto the distal end of the Tuohy-Borst fitting 65 with elastomer sealing ring 68, side port 67 and proximal hub 66. The proximal luer fitting 64 with lumen 69 for balloon inflation is attached to the guide wire tube 51 and the plastic tube 44. The lumen 69 is in fluid communication with the balloon inflation lumen 49 located between the plastic tube 44 and the guide wire tube 51. With the Tuohy-Borst fitting 65 tightened over the plastic tube 44, no fluid such as blood will leak out of the system 40. When the Tuohy-Borst fitting is loosened and slid in the proximal direction it will cause the sheath 42 to slide back releasing the wire basket 52 and filter 53 of FIG. 3A. Subsequent proximal movement of the Tuohy-Borst 65 and sheath 42 will slide back over the ring 57 and collapse the wire basket 52 so the system 40 can be removed from the body.

FIG. 3C shows a longitudinal cross section of central portion of the system 40 when it is built as a rapid exchange device. This section is typically between 5 and 40 cm proximal to the distal end of the system 40. In this section the guide wire tube 51, exits from inside the plastic tube 44 allowing the guide wire 50 to be outside of the catheter 40 in the proximal direction. FIG. 3C show the configuration of the sheath 42 after it has been fully retracted in the proximal direction with the slot 54 in the sheath extending from just distal to the exit of the guide wire tube 51. The slot must be as long or slightly longer than the length needed to slide the sheath back and forth. Like most rapid exchange balloon catheters, plastic tube 44 is attached to a proximal hypotube 14 with balloon inflation lumen 19. In this way, the proximal end of this rapid exchange version is essentially identical to that of the fixed wire system 10 shown in FIG. 2C, with the sheath having the number 42 instead of 12.

FIGS. 4A through 4G are longitudinal cross sections that show the steps in deployment of the system 10 of FIG. 1 to treat an obstructed carotid artery of a human. In FIG. 4A the system 10 has exited the guiding catheter or long introducer sheath (not shown) and is positioned at the site of a stenosis in a vessel of a human body. The stent proximal and distal marker bands 34 and 31 respectively are used to locate the stent 30 with its center placed at the center of the stenosis.

FIG. 4B is a longitudinal cross section of the system 10 of FIG. 1 after the sheath 12 has been pulled back enough to deploy the wire basket 20′ with distal protection filter 22′. This configuration completely filters the blood distal to the stent 30 through the filter 22′ and should catch any distal emboli that result from stent deployment or balloon dilatation.

FIG. 4C is a longitudinal cross section of the system 10 of FIG. 1 after the sheath 12 has been pulled back completely, the stent 30′ has been deployed.

FIG. 4D is a longitudinal cross section of the system 10 of FIG. 1 after the balloon 25′ with control bands 26′ and 29′ has been inflated to post-dilate the stent 30″.

FIG. 4E is a longitudinal cross section of the system 10 of FIG. 1 after the balloon 25 has been deflated and refolded by the control bands 26 and 29 and the sheath 12 has been advanced until it is over the ring 27 and positioned so that additional distal movement of the sheath 12 will collapse the wire basket 20′ with filter 22′.

FIG. 4F is a longitudinal cross section of the system 10 of FIG. 1 before removal from the body but after the radiopaque metallic band 11 of the sheath 12 has been advanced until it engages the distal nose piece 18 so that the wire basket 20 with filter 22 have been completely collapsed.

FIG. 4G shows the artery with expanded stent 30″ after the system 10 has been removed.

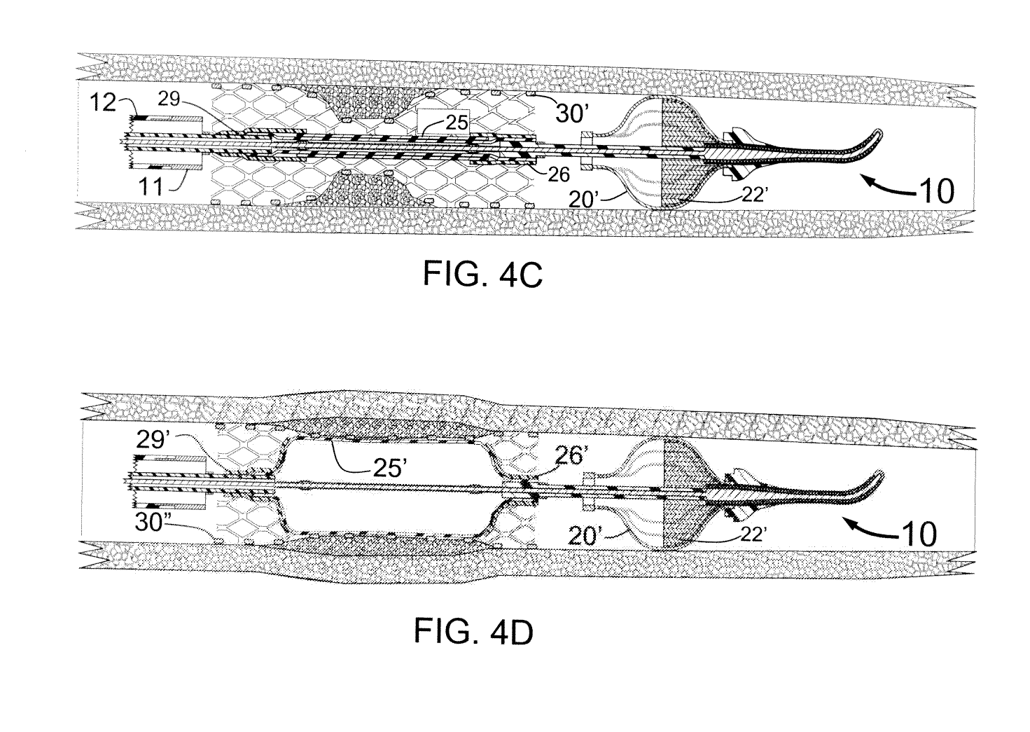

FIG. 5 is a longitudinal cross section of the distal end of the present invention system 70 for the treatment of obstructed vessels of the human body that includes an angioplasty balloon 85 with integrated distal protection. The system 80 includes an integral fixed guide wire 75 with core wire 76 having a distal tapered section 73. The system 70 includes a deployment sheath 72 with metal radiopaque distal end 71 that fits over the distal nose piece 78 which is coaxially attached to the fixed guide wire 75. Under the sheath 72 at the just proximal to the distal nose piece 78 is an expandable embolic filter 82 attached to an expandable/retractable wire basket 80 whose distal end is in turn attached to a radiopaque ring 88. The balloon 85 is located proximal to the radiopaque ring 88 at the proximal end of the wire basket 80. Elastic distal balloon control band 86 and proximal balloon control band 89 placed coaxially over the ends of the balloon 85 act to refold the angioplasty balloon 85 after inflation. The distal end of the balloon 85 is coaxially mounted onto the proximal end of the plastic member 83 that is coaxially located over the core wire 76 proximal to the distal nose 78. The proximal end of the balloon 85 is coaxially mounted onto the distal end of the plastic tube 81. The proximal end of the plastic tube 81 is coaxially mounted onto the hypotube 74 with balloon inflation lumen 79. Radiopaque marker bands 84 and 87 are coaxially mounted onto the core wire 76 at locations corresponding to the ends of the cylindrical portions of the balloon 85 when inflated. An optional heat shrink tube 77 is placed over the radiopaque marker bands 84 and 87 and the core wire 76 so as to prevent the inside of the balloon 85 from coming into contact with the marker bands 84 and 87. The proximal end of the core wire 76 is attached (typically welded) to the hypotube 74. The proximal end of the system 70 is identical to that of the system 10 of FIG. 1 whose proximal end is shown in FIG. 2C.

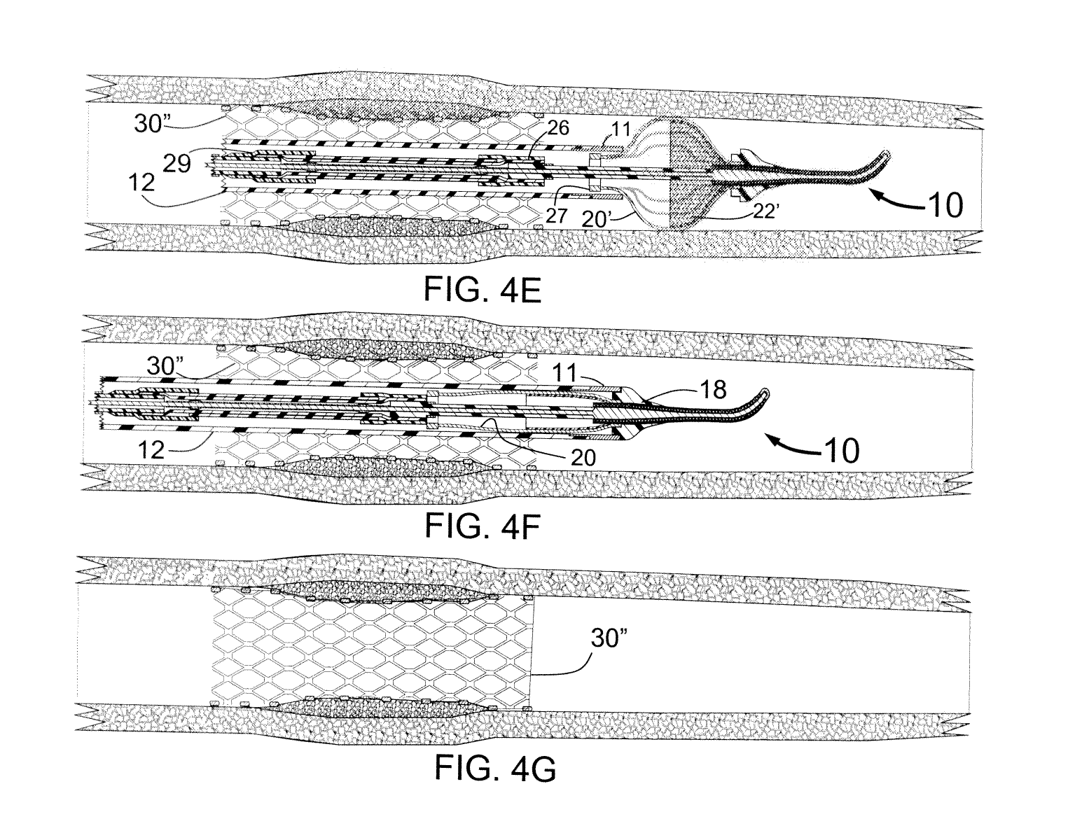

FIG. 6 is a longitudinal cross section of the distal section of a balloon angioplasty catheter 90 with integrated distal protection that is delivered over a guide wire 50. The system 90 with includes a sheath 92 with radiopaque metallic distal end 91 that fits over the distal nose piece 98 that is coaxially mounted over the guide wire tube 94. The system 90 includes an angioplasty balloon 105. Under the sheath 92 just proxial to the distal nose piece 98 is an expandable embolic filter 102 attached to an expandable/retractable wire basket 100 whose distal end is in turn attached to a radiopaque ring 108. The balloon 105 is located proximal to the radiopaque ring 108 at the proximal end of the wire basket 100. Elastic distal balloon control band 106 and proximal balloon control band 109 placed coaxially over the ends of the balloon 105 act to refold the angioplasty balloon 105 after inflation. The distal end of the balloon 105 is mounted onto the guide wire tube 94. The proximal end of the balloon is coaxially mounted onto the distal end of the plastic tube 93. The space between the plastic tube 93 and the guide wire tube 44 is the balloon inflation lumen 99. Proximal and distal radiopaque marker bands 107 and 104 respectively mark the ends of the cylindrical portion of the balloon 104. The system 90 can be either an over the wire or rapid exchange type system as was shown for the system 40 of FIG. 3A.

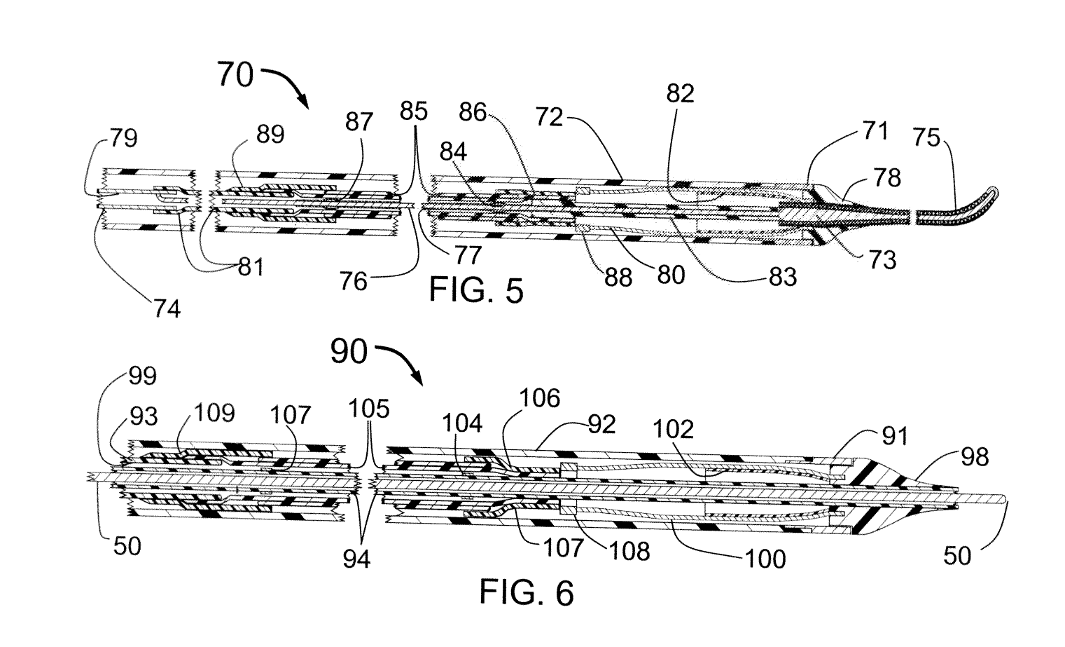

FIG. 7 is a longitudinal cross section of the distal end of the present invention system 110 for the treatment of obstructed vessels of the human body that includes a self expanding stent 130 with integrated distal protection. The system 110 includes an integral fixed guide wire 115 with core wire 116. The proximal section of the core wire 116 is covered by a plastic tube 123. The system 110 includes a plastic deployment sheath 11 that fits over the distal nose piece 118 which is coaxially attached to the fixed guide wire 115. Under the sheath 112 just proximal to the distal nose piece 118 is an expandable embolic filter 122 attached to an expandable/retractable wire basket 120 whose distal end is in turn attached to a radiopaque ring 128. The self expanding stent 130 is located proximal to the radiopaque ring 118 at the proximal end of the wire basket 120. Radiopaque marker bands 124 and 127 are coaxially mounted onto the plastic tube 123 at locations corresponding to the ends of the stent 130. The proximal end of the system 110 includes a mechanism similar to that of FIG. 2C where the hypotube is replaced by the tube 123.

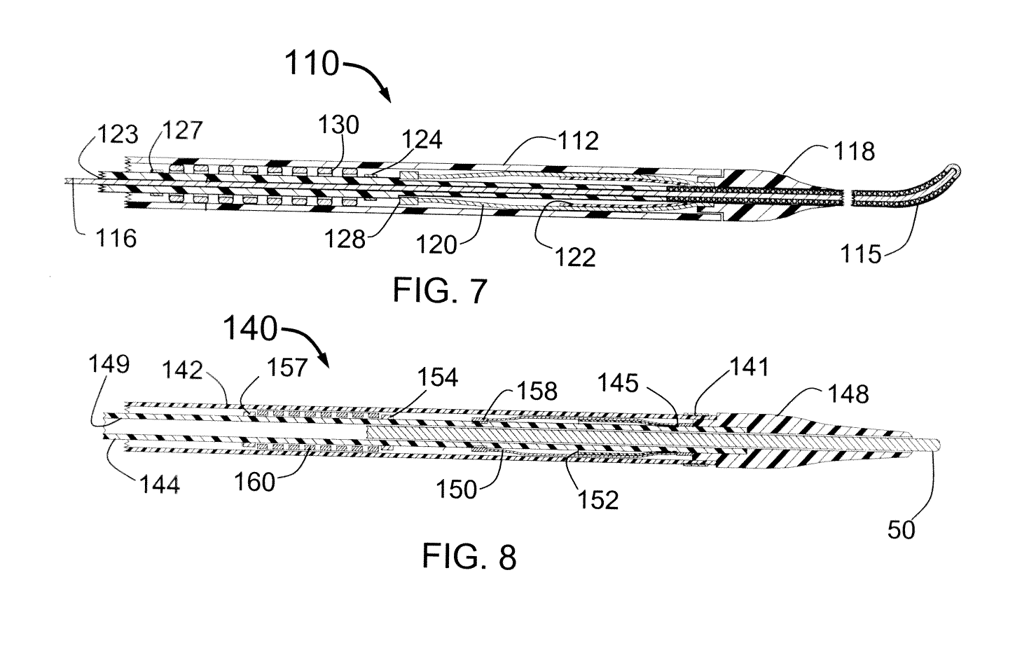

FIG. 8 is a longitudinal cross section of the distal section of a self expanding stent delivery system 140 with integrated distal protection that is delivered over a guide wire 50. The system 140 with includes a sheath 142 with radiopaque metallic distal marker band 141 that fits over the distal nose piece 148 that is coaxially mounted over the guide wire tube 144 with guide wire lumen 149. The system 140 includes a self expanding stent 160. Under the sheath 142 at the just proximal to the distal nose piece 148 is an expandable embolic filter 152 attached to an expandable/retractable wire basket 150 whose distal end is in turn attached to a radiopaque ring 158. An additional radiopaque marker band 145 is coaxially mounted onto the guide wire tube 144 just proximal to the nose piece 148. The two markers 141 on the sheath and 145 on the guide wire tube allow visualization as to when the sheath is fully advanced in the distal directions which clearly indicates that the filter basket 150 has been fully collapsed. This approach using two marker bands could also be applied to the systems shown in FIGS. 1 through 7. The self expanding stent 160 is located proximal to the radiopaque ring 158 at the proximal end of the wire basket 150. Proximal and distal radiopaque marker bands 157 and 154 respectively mark the ends of the stent 160. The system 140 can be either an over the wire or rapid exchange type system as was shown for the system 40 of FIG. 3A.

It is envisioned that the filter basket may be made of a radiopaque material such as tantalum or an alloy with embedded tungsten such as L605 so as to make visualization of the expansion and retraction of the filter basket and filter easier under fluoroscopy. It is also envisioned that if the filter basket is made of a memory metal such as NITINOL then it may have one or more radiopaque markers placed on or inside it such as a gold marker. It is also envisioned that the entire filter basket might be electroplated with a radiopaque metal such as gold or platinum.

In all these examples is should be clear that any of the configurations can be developed to be over the wire, rapid exchange or fixed wire systems. The self expanding stents would typically be made of NITINOL while the balloon expandable stents would typically be made from 316L surgical grade stainless steel, L605, another suitable cobalt chromium alloy or a layered metallic tube having at least one layer of a radiopaque metal such as tantalum. Any of the stents may also be drug eluting using one or more drugs. Typical drugs would include the -imus drugs of Sirolimus and Everolimus. The stents may also be carbon coated to reduce sub acute thrombosis.

It is envisioned that the self expanding stent versions of the present invention would be ideally suited to carotid arteries and above the knee SFA and Popliteal arteries. The balloon expandable version of the present invention would have best application to the treatment of myocardial infarction.

Various other modifications, adaptations, and alternative designs are of course possible in light of the above teachings. Therefore, it should be understood at this time that within the scope of the appended claims the invention may be practiced otherwise than as specifically described herein.

What is claimed is:

1. A percutaneous system for opening a stenosed vessel of a mammal comprising:
(a) a catheter for insertion into a mammalian vessel having a stenosis, said catheter having a proximal end section and a distal end section;
(b) an expandable filter mechanism mounted inside a deployable sheath for radial expansion against a wall of the vessel when the sheath is displaced by a first displacement distance, the filter mechanism when expanded being capable of capturing embolic material released from said stenosis;
(c) a stenosis opening mechanism mounted inside the deployable sheath proximal to the expandable filter mechanism of the catheter, the stenosis opening mechanism being radially expandable against the stenosis when the stenosis opening mechanism is positioned within the vessel in longitudinal alignment with the stenosis following displacement of the sheath by a second displacement distance; and
(d) radiopaque markers for aligning the stenosis opening mechanism with the stenosis.

2. The percutaneous system as recited in claim 1, wherein the expandable filter mechanism includes a filter mechanism positioning marker element for positioning the expandable filter mechanism distal to the stenosis in the vessel.

3. The percutaneous system as recited in claim 1, wherein the expandable lifter mechanism is fixedly mounted to a distal nosepiece of the catheter at a first end thereof.

4. The percutaneous system as recited in claim 2, wherein the filter mechanism positioning element is fixedly mounted to the expandable filter mechanism at a second end thereof.

5. The percutaneous system as recited in claim 2, wherein the expandable filter mechanism includes a reversibly expandable wire structure contoured into a basket structure when expanded.

6. The percutaneous system as recited in claim 5, including an embolic filter material composition fixed to at least a position of an inner wall of the basket structure for capturing the embolic material.

7. The percutaneous system as recited in claim 6, wherein the embolic filter material composition is porous.

8. The percutaneous system as recited in claim 7, wherein the embolic filter composition includes anti-thrombogenic properties.

9. The percutaneous system as recited in claim 1, wherein the stenosis opening mechanism includes:
(a) a radially expandable stent mounted within the sheath of said catheter for radial expansion of the stent subsequent to longitudinal alignment of said stent with the stenosis and displacement of said sheath by the second displacement distance to permit exposure of the stent to said stenosis; and
(b) a balloon mounted to the catheter in longitudinal alignment with the stent for radially displacing the stent when the balloon is inflated, the balloon being in fluid communication with a balloon inflation lumen located within the catheter proximal to the balloon, the balloon being radially expandable by introduction of a fluid through the balloon inflation lumen.

10. The percutaneous system as recited in claim 9, wherein the stent is a balloon expandable stent that will expand radially when the sheath is displaced by the second displacement distance uncovering the stent, and the balloon is inflated to expand the stent.

11. The percutaneous system as recited in claim 9, wherein the stent is a self-expanding stent that will expand radially when the sheath is displaced by the second displacement distance thereby uncovering and releasing the self-expanding stent, the balloon being inflated following stent expansion to further expand the stent within the stenosis.

12. The percutaneous system as recited in claim 1, wherein the stenosis opening mechanism is a self-expanding stent that will expand radially when the sheath is displaced by the second displacement distance thereby uncovering and releasing the self-expanding stent.

13. The percutaneous system as recited in claim 1, wherein the stenosis opening mechanism is an angioplasty balloon fixed to the catheter proximal to the filter mechanism, the balloon being in fluid communication with a balloon inflation lumen located within the catheter proximal to the balloon, the balloon being radially expandable against the stenosis when the balloon is aligned with the stenosis, the sheath is displaced by the second displacement distance thereby uncovering the balloon and the balloon is inflated by introduction of a fluid through the balloon inflation lumen.

14. A catheter for opening a stenosed carotid artery including:
a guide wire attached to the catheter and extending in a distal direction, the guide wire being the distal end of the catheter;
an embolic filter located on the catheter proximal to the guide wire forming the distal end of the catheter, the embolic filter being expandable within the carotid artery;
a self expanding stent located proximal to the embolic filter;
an angioplasty balloon having a length that is less than the stent, the balloon being positioned on the catheter in the center of the stent; the balloon being in fluid communication with an inflation lumen extending from the proximal end of the balloon to the proximal end or the catheter, the balloon being designed to be used for post-dilation of the stent;
balloon control bands coaxially positioned over the most proximal and most distal sections of the balloon, the control bands being designed to constrain the ends of the balloon when inflated and to compress the balloon following balloon deflation,
a sheath in the form of a thin wall tube that is positioned coaxially over the stent, balloon and embolic filter, the distal end of the sheath being initially distal to the embolic filter, the proximal end of the sheath being located near the proximal end of the catheter and having means to move the sheath in the proximal and distal directions, the sheath being designed to constrain the embolic filter and self expanding stent so that when the sheath is pulled in the proximal direction, the embolic filter is first released, the self expanding stent is then released, the sheath then being advanced in the distal direction will first cover the angioplasty balloon and then will collapse the embolic filter so that the catheter can then be removed from the body.
